(19) United States
(12) Patent Application Publication
(10) Pub. No.: US 2002/0127603 A1
(43) Pub. Date: Sep. 12, 2002

(54) Methods for synthesizing reporter labeled beads

(75) Inventors: David A. Basiji, Seattle, WA (US); William E. Ortyn, Bainbridge Island, WA (US)

Correspondence Address: LAW OFFICES OF RONALD M ANDERSON, 600 108TH AVE, NE, SUITE 507, BELLEVUE, WA, 98004, US

(73) Assignee: Amnis Corporation

(21) Appl. No.: 09/976,238

(22) Filed: Oct. 12, 2001

Related U.S. Application Data
(60) Provisional application No. 60/242,734, filed on Oct. 23, 2000.
(60) Provisional application No. 60/240,125, filed on Oct. 12, 2000.

(51) Int. Cl. C12Q 1/68; G01N 33/53; G01N 33/543

(52) U.S. Cl. 435/7.1; 436/518

(57) ABSTRACT

Methods for constructing reporter labeled carriers (such as beads) using a plurality of optically distinguishable carriers for chemical synthesis or attachment, such that the number of unique reporters required to label a carrier is reduced. One embodiment employs carriers that themselves have optically distinguishing characteristics. A carrier's identity is encoded by the combination of the optical characteristics of its reporter set, as well as the optical characteristics of the carrier itself. In other embodiments, different reporters are discriminable based on the intensity of their color labels, their size, and/or other optically detectable characteristics, and not necessarily by the presence or absence of particular colors. Another embodiment is directed to generating a plurality of reporters from a plurality of singly labeled micro-particles. The present invention can be employed in conjunction with a split/add/pool (SAP) or a directed synthesis process.

### RELATED APPLICATIONS

This application is based on prior co-pending provisional application Serial No. 60/240,125, filed on Oct. 12, 2000, and prior co-pending provisional application Serial No. 60/242,734, filed on Oct. 23, 2000, the benefit of the filing dates of which are hereby claimed under 35 U.S.C. §119(e).

### FIELD OF THE INVENTION

The present invention generally relates to a method and apparatus employed to optically encode large libraries of particles to discriminate particle-bound molecules from each other, including particles used as substrates for deoxyribonucleic acid (DNA) oligomers, polypeptides, drug candidates, antibodies, and other molecular entities for which it is advantageous to assay a wide diversity of entities, and more specifically, relates to the generation of encoded bead libraries, preferably to be analyzed using an imaging system employing spectral decomposition and preferably accomplished with the beads in flow.

### BACKGROUND OF THE INVENTION

Two methods of encoding particle libraries in the prior art call for the placement of optically distinguishable reporters on a population of solid supports during combinatorial chemical synthesis. The attachment of reporters to the solid supports may be by means of covalent bonds or colloidal forces. The solid supports (“carriers”) are typically beads of polystyrene, silica, resin, or any another substance on which compounds can be readily synthesized, generally in a size range of ten to several hundreds of microns in diameter. The reporters are typically beads of similar material, but much smaller than the carriers, to accommodate the attachment of numerous different reporters to the larger carriers. The identity of each carrier is encoded by its unique combination of associated reporters each of which has a distinct optical characteristic.

In the prior art, reporter-based optical encoding is performed in a split/add/pool (SAP) combinatorial process in parallel with the synthesis of chemical compounds on the surface of the carriers. During this procedure, a unique reporter is attached to each carrier in conjunction with the chemical addition or modification that is co-executed at each step in the SAP process. Each reporter thereby encodes both the synthetic operation as well as its place in the synthetic process. Such an SAP combinatorial process in parallel with the synthesis of chemical compounds is described in U.S. Pat. No. 5,708,153, entitled “Method of Synthesizing Diverse Collections of Tagged Compounds,” filed on Jun. 7, 1995, and issued on Jan. 13, 1998, the disclosure and drawings of which are hereby specifically incorporated herein by reference, for purposes of providing background information regarding the SAP process.

By enumerating the optical characteristics of each reporter on a carrier, it is possible to synthesize libraries of unique compounds numbering in the billions. For example, numerous useful genetic assays can be performed by combinatorially synthesizing oligonucleotides on a carrier library such that a given carrier bears numerous identical covalently bound oligos and each carrier in the library bears a different oligo sequence. In addition to its oligo sequence, each carrier bears a unique optical signature comprising a predefined combination of different reporters, where each reporter contains a predefined combination of different fluorochromes. A carrier's optical signature is correlated to the addition sequence of each reporter during the synthetic process to enable identifying the unique nucleotide sequence on that carrier. By imaging the carriers, the optical signatures can be read and correlated to the corresponding oligo sequences. The carriers are used as probes for identifying genomic traits, such as SNP content and DNA sequences, as well as for other applications as outlined below.

Though existing methods excel at producing a large diversity of labeled carriers in a split/pool combinatorial process, these methods have generally been conceived in the absence of specific, optimized means for imaging and analyzing the optical signatures on each carrier and of the library as a whole. However, exemplary means for carrying out these functions are disclosed in flow imaging systems described in applicants' above-referenced previously filed U.S. provisional patent application, Serial No. 60/240,125, entitled “Method And Apparatus for Synthesizing and Reading Reporter Labeled Beads.” When the process of imaging reporter-labeled carriers is taken into account, the limitations in the prior art of reporter-labeled carrier synthesis become evident.

One limitation of the prior art is the need for large numbers of reporters on each carrier. This limitation is due both to the need for as many as ten or more reporter types to encode an equivalent number of co-executed chemical synthetic steps, as well as the requirement that each reporter type be present in multiple copies on the surface of the carrier to ensure uniform coverage of the carrier surface. At least one of each type of reporter on a carrier must be in view during the imaging process in order to successfully decode the carrier's signature. Since reporters are randomly distributed over the carrier surface, it is possible and even likely that a given reporter will be out of view when the carrier is imaged, preventing the accurate identification of the carrier. This problem can be addressed by attaching multiple copies of each reporter to the bead, thereby increasing the odds that at least one reporter of each type will lie in view. However, reporter redundancy is constrained by the need to maintain a significant fraction of exposed carrier bead surface for molecular synthesis or attachment, and high reporter redundancy increases the complexity of carrier image analysis. Hence, there exists a need for an encoding scheme that minimizes the number of reporters per carrier.

Another limitation of the prior art is the necessity of employing many colors to produce a sufficiently large library of reporter types. Existing reporter-labeled carrier encoding schemes typically employ binary color-coded reporters, wherein each reporter type is defined by a unique combination of colors. Binary reporter coding requires a large number of colors (e.g., six different fluorescent dyes or quantum dots are required to produce a set of 40 reporters necessary to encode all possible DNA 10-mers). The need to analyze large numbers of colors greatly increases instrument complexity. Thus, there is a need for an encoding scheme that minimizes the number of colors per reporter.

Still another limitation of the prior art is the monolithic structure of the reporters themselves. Reporters containing multiple fluorescent dyes in a homogeneous mixture can be subject to dye interactions such as fluorescence resonant energy transfer and self-filtering that alter the observed color code of a reporter. Such phenomena are exacerbated by spectral overlap between dyes due to the use of large numbers of colors. Thus, there is a need for a reporter structure that minimizes interactions between color signals.

Yet another limitation of the prior art is the use of an SAP process for the attachment of reporters to carriers. An SAP process results in the final pooling of all carriers, thereby preventing the subsequent synthesis or chemical attachment of compounds to specific carriers. Combining compound synthesis and carrier encoding in a single process makes it difficult to prevent interference between the synthetic chemistry and the physical or chemical linking of reporters to the carrier. Likewise, the coating of an exposed carrier surface by chemical synthesis intermediates can interfere with or completely block reporter attachment. Even if the hurdles of co-execution are overcome, the final result is still a pooling of all carriers in the library, thereby preventing the selection of library subsets for faster analysis and better hybridization kinetics. Hence, there is a need for a method of generating encoded substrates that is independent of the synthesis or attachment of chemical compounds to those substrates, and which can be performed without a final pooling of the substrates.

### SUMMARY OF THE INVENTION

The present invention is directed to a method of constructing a library of optically distinct reporter labeled carriers. One advantage of the present invention is that it reduces the number of reporters necessary to encode a library of carriers by employing optically distinguishing characteristics for the carriers themselves. A carrier's identity is encoded by the combination of the optical characteristics of its reporter set as well as the optical characteristics of the carrier itself, thereby reducing the number of reporters necessary to encode a library of a given complexity.

Another advantage of the present invention is the discrimination of different reporters based on the intensity of their color labels, their size, or other optically detectable characteristics, an not just in response to the presence or absence of particular colors. By using intensity and other parameters, the number of colors necessary to encode a set of reporters can be greatly reduced. Such reporters can be incorporated into an SAP or directed synthesis process to encode carriers.

A further aspect of the invention is directed to a novel method of generating a plurality of reporters from a plurality of singly labeled micro-particles. Each singly labeled micro-particle comprises a uniquely identifiable optical characteristic, such as the emission of a particular color, but is below the resolution limit of the imaging system used to analyze the carrier library. A set of unique reporters is generated by combining different singly labeled micro-particles into aggregates, each aggregate acting as a single reporter having a combination of optical characteristics determined by the aggregation of micro-particles. In this manner, reporters with complex optical properties can be generated from relatively simple micro-particles.

Still another aspect of the present invention provides for the directed synthesis of chemical compounds on carriers in conjunction with the generation of reporter signatures on those carriers in a plurality of reaction vessels such that each unique carrier occupies a dedicated vessel. In this manner, subsets of the carrier library can be easily assembled by combining isolated carriers from a specific set of vessels.

In still another aspect of the invention, reporter labeled carriers are produced in a single-step reaction in a plurality of reaction vessels such that each unique carrier occupies a dedicated vessel. In this aspect of the invention, chemical synthesis on, or chemical addition to, each carrier is performed subsequent to the production of the carrier library itself. In this manner, physical and chemical processes employed during carrier library generation are separate from the processes of chemical compound synthesis or chemical attachment to the carriers, while still preserving the ability to assemble subsets of the carrier library by combining isolated carriers from a specific set of vessels.

It is contemplated that the present invention will be applied to carriers and compounds, created by combinatorial SAP synthesis, as well as to specifically directed synthesis of carriers and compounds, and to compounds synthesized or attached to pre-encoded carriers.

### BRIEF DESCRIPTION OF THE DRAWING FIGURES

The foregoing aspects and many of the attendant advantages of this invention will become more readily appreciated as the same becomes better understood by reference to the following detailed description, when taken in conjunction with the accompanying drawings, wherein:

FIG. 1 (Prior Art) is a schematic illustration showing an exemplary SAP combinatorial synthesis scheme for the synthesis of bound oligonucleotides and the generation of the corresponding optical reporter signatures on a plurality of carriers;

FIG. 2 is a schematic illustration showing for one example, the number of unique pairs and unique binary codes represented with N unique reporter colors;

FIG. 3 is a schematic illustration showing an exemplary SAP combinatorial synthesis scheme for the synthesis of bound oligonucleotides and the generation of the corresponding intensity-coded optical reporter signatures on a plurality of carriers;

FIG. 4 is a schematic illustration showing a second exemplary SAP combinatorial synthesis scheme for the synthesis of bound oligonucleotides and the generation of the corresponding intensity- and size coded optical reporter signatures on a plurality of carriers;

FIG. 5 is a schematic illustration of an example in which the carrier is itself optically distinguishable based on color;

FIG. 6 is a schematic illustration showing the subset of trajectories from the SAP scheme of FIG. 1 necessary to produce all DNA tetramers specifically beginning with “A,” ending with “T,” and having either a “G” or “C” in the third position;

FIG. 7 is a schematic illustration showing how the example specific DNA library of FIG. 6 can be encoded with only one unique reporter bound to each carrier in a constrained SAP process;

FIG. 8 is a schematic illustration showing how the example specific DNA library of FIG. 6 can be encoded with only one unique reporter bound to each carrier in a directed synthesis in discrete reaction vessels;

FIG. 9 is a schematic illustration showing how the example specific DNA library of FIG. 6 can be generated on previously encoded carriers;

FIG. 10 is a schematic illustration of the spectral decomposition scheme by which reporter-labeled carriers are decoded when the carriers are not optically distinguishable from each other;

FIG. 11 is a schematic illustration of the same reporter colors for each carrier as in FIG. 10, but encoded in accord with the present invention, wherein the color of the carrier itself serves to partially identify the carrier;

FIG. 12 is a schematic illustration showing the use of carriers that employ size as an encoding parameter in addition to the bound color-coded reporters of the previous examples;

FIG. 13 is a schematic illustration showing images that are projected onto a detector for the spectral decomposition embodiment when three carriers are in view; and

FIG. 14 is a schematic illustration of a method for combining four color species of singly-labeled microbeads to produce all possible binary color codes in 2⁴ reaction vessels.

### DESCRIPTION OF THE PREFERRED EMBODIMENT

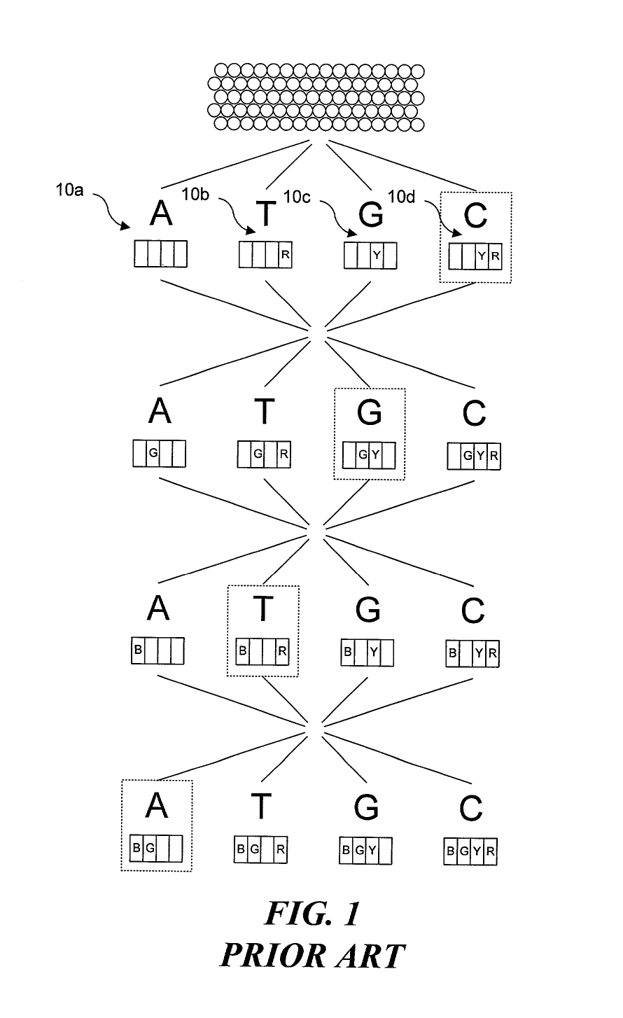

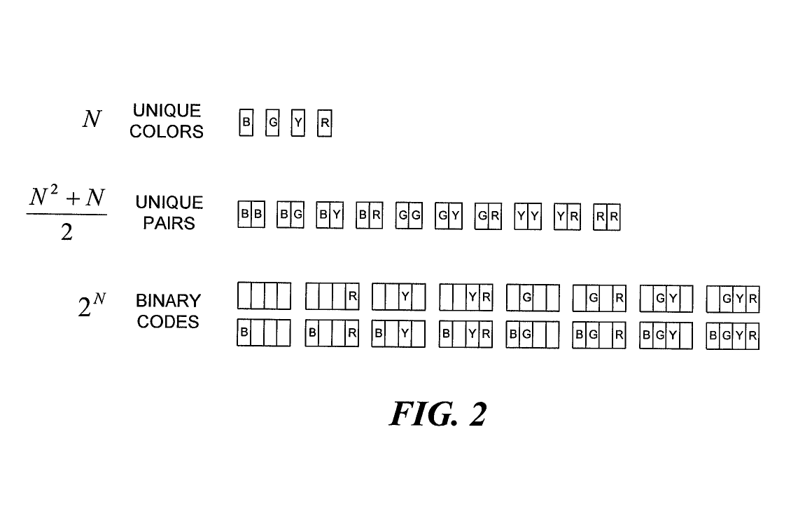

In prior art reporter-labeled carrier encoding, the identity of a carrier is determined by the combination of different reporter types on the carrier, as produced in an SAP process. The reporter types, and therefore the carrier identities, are defined in the prior art by the combination of colors present or absent on each reporter. FIG. 1 illustrates the synthesis of DNA tetramers 10a, 10b, 10c, and 10d on carriers using an SAP process of the prior art. The reporters shown in FIG. 1 form a binary code of four digits, one per color, where each color is either present or absent. Since the SAP synthetic matrix of FIG. 1 has sixteen nodes, and a unique reporter is required for each node, at least four colors are necessary to produce a sufficiently large set of reporters. As illustrated in FIG. 2, there are a number of different reporter identities that can be generated based on the presence or absence of different colors on the reporter. The simplest encoding scheme employs a unique color per reporter type. If colors are combined in unique pairs, more reporter types can be generated. If all colors can be independently present or absent on a reporter, the result is a true binary code. The number of unique carrier signatures, N, that can be created using R reporters comprising some combination of C colors in a binary encoding scheme is as follows:

$$N = {( \frac{{2}^{C}}{R} )}^{R} \qquad ( 1 )$$

The numerator of the fraction is the number of different reporter types that can be produced for a given number of colors. Because the total number of unique carriers that can be generated is an exponential function of the number of reporter types, the total number of unique carriers that can be created is quite substantial. For example, using six colors and ten reporter types per carrier results in a carrier library of over 115 million combinations, while using eight colors and sixteen reporter types per carrier results in libraries that can exceed 1.8×10¹⁹ possible combinations. There are numerous potential applications of large compound libraries, including DNA sequencing, genotyping, immuno-phenotyping, but such applications remain impractical without the present invention, which is a different manner of encoding reporter-labeled libraries to facilitate their analysis.

Reporter Color Conservation

One aspect of the present invention serves to reduce the number of colors necessary to generate a carrier library of a given size by increasing the number of different reporter types that can be generated using a given number of colors. The cost and complexity of a carrier analysis system is a strong function of the number of colors necessary to encode a carrier library. If the colors are generated by fluorescent dyes, additional excitation light sources, excitation filtering, collection filtering, and crosstalk correction are required. In the present invention, reporters are discriminated using information that can include size, shape, color intensity, or other optically distinguishable properties, either alone or in combination. Unlike the prior art, the reporters of the present invention can be employed to encode carriers in directed synthesis, constrained SAP synthesis, or in the absence of any chemical synthesis.

A preferred embodiment of the present invention employs intensity coding instead of a simple binary color encoding. With the substitution of intensity coding for binary coding, the “2”in equation (1) is replaced by I, the number of intensities that can be generated for a given color:

$$N = {( \frac{{I}^{C}}{R} )}^{R} \qquad ( 2 )$$

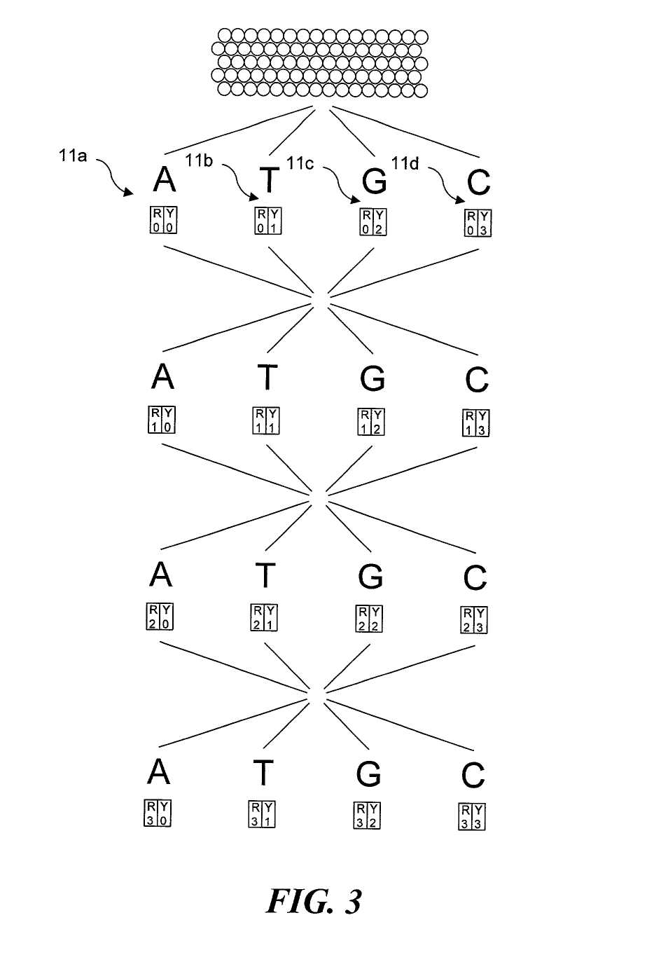

By employing even modest intensity coding, the number of colors employed to generate the libraries in the examples above can be greatly reduced, simplifying the design of analysis instrumentation. This is illustrated in FIG. 3, where the same SAP synthesis as FIG. 1 is demonstrated using four intensity levels of two colors. In FIG. 3, reporter 11a is encoded by red color R at intensity level 0 combined with yellow color Y at intensity level 0, while reporter 11b is encoded by red color R at intensity level 0 combined with yellow color Y at intensity level 1. The other reporters in the synthetic process are similarly encoded by unique combinations of intensities of the two colors used in the example. In the example of FIG. 3, only half as many colors are necessary to encode the synthesis compared to the non-intensity coded example of FIG. 1.

Revisiting the 10- and 16-reporter examples above, a library of 115 million carriers can be generated using only three colors instead of six if each color is present in four intensity levels. Similarly, a library of 1.8×10¹⁹ unique carriers can be produced with only four colors in four intensity levels each.

Intensity coding of reporters can be accomplished in the present invention by a number of standard means used to label beads, including loading reporter beads with different concentrations of fluorescent or absorbent dye, aggregating different quantities of luminescent particles such as quantum dots into a reporter, or employing different sizes of reporters each containing a given concentration of fluorescent dye such that the total dye content (and therefore the intensity) of a reporter is determined by the size of the reporter. In the latter case, the size of the reporter can be used as an additional discriminating parameter if the various reporter sizes employed exceed the resolution limits of the imaging system used to analyze the carrier library. Under these circumstances, equation (2) is modified with an additional term S, which corresponds to the number of different reporter sizes that can be discriminated:

$$N = {( \frac{{SI}^{C}}{R} )}^{R} \qquad ( 3 )$$

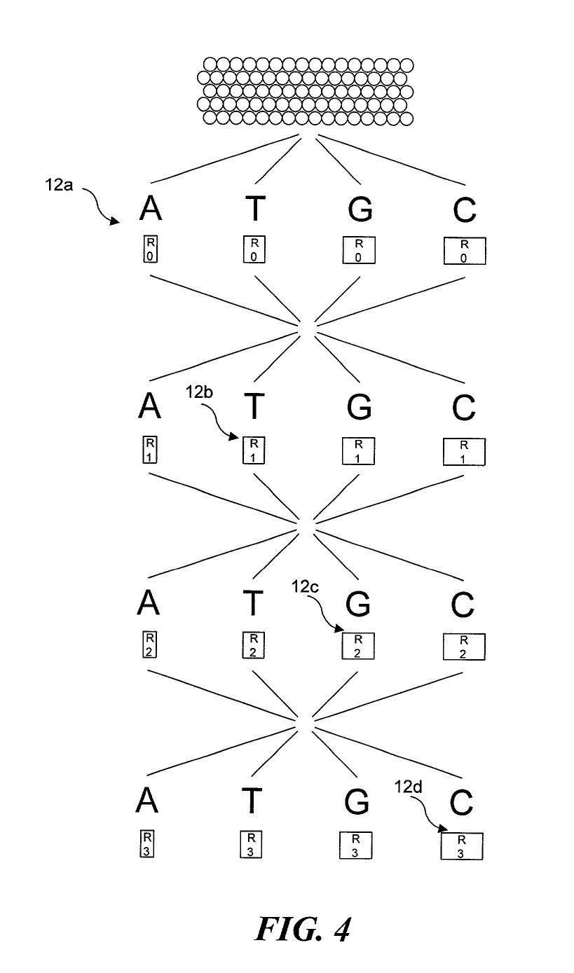

In general, the S term corresponds to the number of different states that can be distinguished from a reporter as a whole, such as different sizes, shapes, or other physical properties. Each additional reporter parameter multiplies the total number of unique reporters that can be produced without increasing the number of colors. FIG. 4 illustrates the use of reporter size as an additional means of generating optically distinct reporters to further reduce the number of colors compared to the examples of both FIG. 1 and FIG. 3.

In FIG. 4, each reporter has a unique combination of four different sizes and intensities. Reporter 12a has intensity 0 of red dye R and is the smallest of four different sized reporters employed. In contrast, reporter 12b has intensity 1 of red dye R and is larger than reporter 12a, but smaller than reporters 12c and 12d. By employing both size and intensity to distinguish reporters, the number of colors employed is halved relative to the example of FIG. 3, and is only one quarter the number of colors employed in the prior art example of FIG. 1. In the case of the 10-reporter carrier construct cited earlier, if four different reporter sizes can be discriminated along with four different intensities of each color, a library of 115 million unique carriers can be generated using only two colors.

Reporter Conservation

Another aspect of the present invention improves on prior art by employing the optical properties of the carriers themselves to partially encode carrier identity. By so doing, the number of unique reporters required to unambiguously encode a carrier is reduced, thereby simplifying the task of image analysis of each carrier and increasing the carrier surface area available for chemical synthesis or attachment. In a prior art SAP synthetic strategy, such as that illustrated for DNA in FIG. 1, the synthetic fate of any given carrier is defined by its trajectory through a synthesis matrix. In the case of DNA synthesis, there are four chemical subunits (A, C, G, and T; the nucleotide bases that are the essential constituents of DNA), corresponding to the width of the matrix. A synthetic matrix for polypeptide synthesis would have a width of 20, corresponding to the number of naturally occurring amino acids. The height of the synthetic matrix in FIG. 1 is simply the number of nucleotide additions necessary to produce the required DNA polymer length, in this example a four-step SAP synthetic process is used to produce all possible DNA tetramers. The number of reporters required to encode a complete SAP synthesis, as illustrated in FIG. 1, is just the matrix width times its height. Any given carrier produced by the synthesis requires a number of reporter types equal to the polymer length. The actual number of reporters on each carrier is the number of reporter types times the redundancy of each reporter type. For example, in a synthesis of DNA ten-mers, at least 40 different reporter types are required and if each reporter is present in 10-fold redundancy, then each carrier will bear an average of 100 individual reporters, of ten different types.

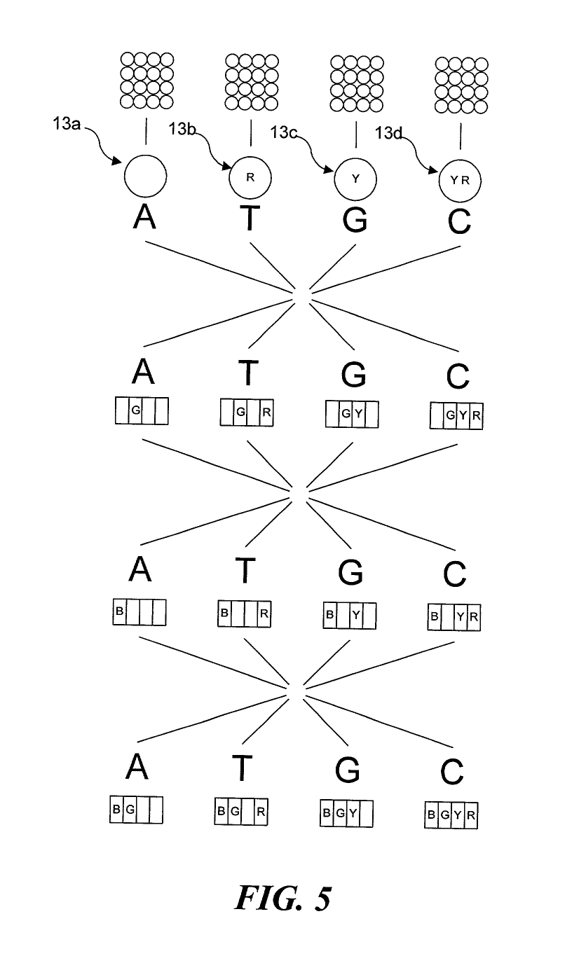

In contrast to the prior art illustrated in FIG. 1, FIG. 5 illustrates the same DNA synthesis performed in a manner of the present invention, wherein the carriers themselves have distinguishable optical characteristics, obviating the need for one or more reporters. As shown in FIG. 5, four distinguishable batches of labeled carriers 13a-13d are used as the starting points for a modified SAP synthetic process, where the first nucleotide addition occurs by directed synthesis, followed by SAP process to synthesize the remaining oligo on each carrier, and to attach reporters. The four distinguishable carrier types are initially in four separate pools rather than one pool, as would be the case in the prior art. For clarity, in FIG. 5 each carrier is fluorescently labeled with the same color codes employed for the first four reporters of FIG. 1: carrier 13a is blank, carrier 13b is labeled with red dye R, carrier 13c is labeled with yellow dye Y, and carrier 13d is labeled with both red dye R and yellow dye Y. However, since the color code is arbitrary, the particular color labels can be any valid color code as desired, or any other optically distinguishable trait.

In a modified SAP process incorporating distinguishable carriers of the present invention, the number of optically distinguishable carrier types can be equal to the width of the synthetic matrix, thereby reducing the number of distinct reporter types attached to each carrier by one. In addition, the present invention can utilize more or fewer distinguishable carrier types than the matrix width. For example, by employing sixteen different carrier types, all possible DNA dimers can be synthesized separately on each carrier in a directed process that occurs in sixteen separate vessels, prior to the execution of an SAP synthesis process and reporter labeling for subsequent DNA extension. Reducing the number of reporter types simplifies image analysis of the carriers, increases the carrier surface area available for chemical synthesis, and allows increased redundancy in the number of copies of each reporter type attached to a carrier, thereby increasing the probability that at least one copy of each reporter will be imaged as is required for identification of a carrier. As this example shows, the distinguishable substrates of the present invention can be employed in either directed synthesis, SAP combinatorial synthesis, or a combination of the two.

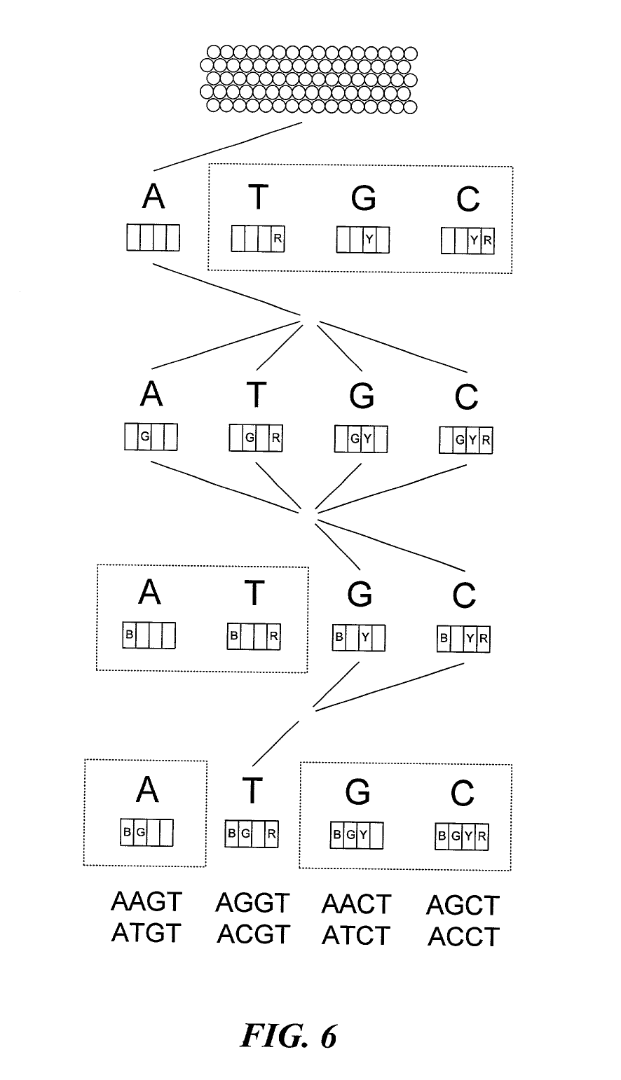

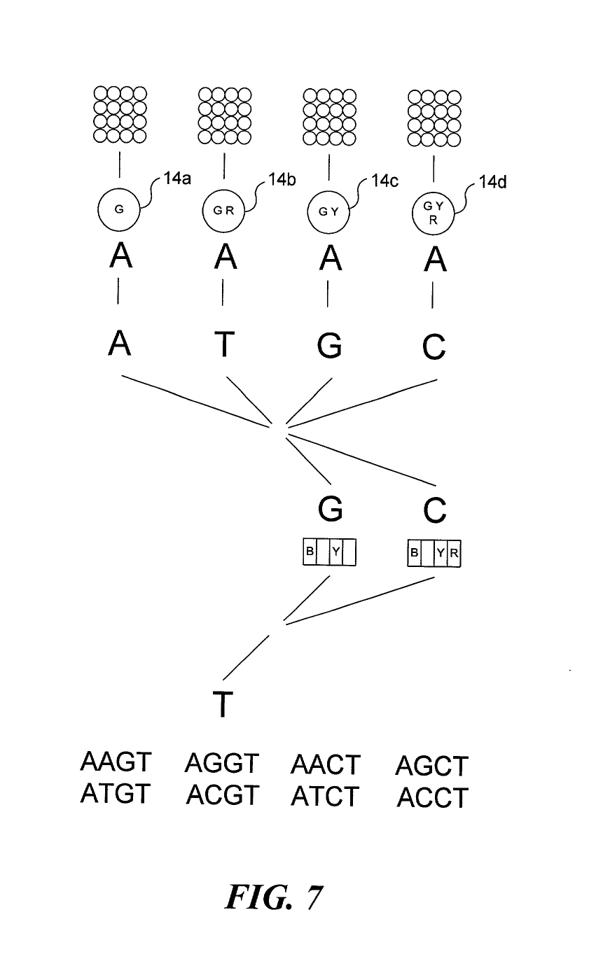

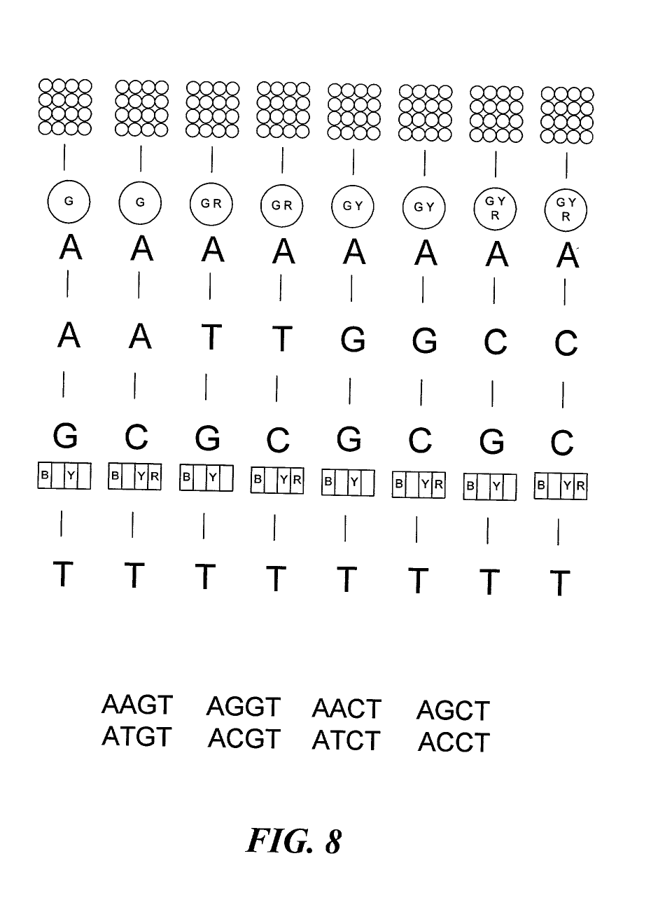

The present invention also reduces the number or reporters necessary to encode a constrained SAP processes or a directed synthesis. An unconstrained SAP synthesis results in carriers following every possible trajectory through the synthetic matrix. FIG. 6 illustrates the subset of trajectories from the SAP scheme of FIG. 1, which is necessary to produce all DNA tetramers beginning with “A”, ending with “T”, and having either a “G” or “C” in the third position. In a constrained SAP process, each splitting step results in only as many pools as are required to produce the molecular diversity necessary for each position in the oligomer. For example, FIG. 6 shows that the first base of each desired DNA oligo is an “A,” so there is no splitting of the carriers prior to the addition of the first “A.” The second oligo position can contain any base, so the carriers are split into four separate reactions (one for each nucleotide) prior to addition of the second base. The third nucleotide can be either a “C” or a “G,” so the carriers are pooled and split into only two reactions for the third nucleotide. Finally, since the last nucleotide is always a “T,” the final nucleotide is added to all the carriers. In the prior art, every synthetic step is associated with a reporter addition, whereas in the present invention, there is no need to add a reporter to the carrier to encode the first and last base positions of this example, thereby reducing the number of reporters per carrier. Further, since each carrier can be optically distinguished in the present invention, they can be labeled as necessary to encode the second nucleotide position. Therefore, the example library of FIG. 6 can be encoded with the process shown in FIG. 7, whereby only one unique reporter is bound to each carrier. The carriers are kept isolated from each other until after the addition of their respective bases, at which point, they are pooled and split as necessary for the subsequent nucleotide addition and reporter binding steps. As illustrated in FIG. 8, which is a directed synthesis of the same DNA oligonucleotides shown in FIG. 7, the use of optically distinct carriers 14a-14d and the omission of reporters from synthetic steps in the present invention can also be extended to directed synthesis.

In FIG. 8, each distinct reporter-labeled carrier and oligo species is synthesized in a step-wise fashion in separate reaction vessels. As in FIG. 7, the eight different carrier types are employed to encode the first two oligo positions (of which there are eight different combinations) and the addition of a single reporter type to each carrier occurs only to encode the difference between a “C” or “G” nucleotide in the third position of the oligo. Since the fourth position of every oligo is a “T”, no reporter is required to distinguish the identity of the nucleotide at that position. Directed synthesis in the present invention offers a significant advantage over SAP synthesis of the prior art in that the encoded carriers are not pooled during the synthetic process, allowing specific carrier subsets to be assembled from the larger set of carriers to speed sample analysis. In one example, every possible DNA oligo of length 10 can be synthesized in a directed manner on approximately one million encoded carriers. However, only a small fraction of this total library may be necessary to sequence or genotype a specific gene from an individual DNA sample. Based on knowledge of the nominal gene sequence, a subset of the complete DNA carrier library can be assembled and hybridized to the DNA of interest. Since most genes are on the order of 1000 nucleotides in length, it is expected that the number of carriers in the subset would be approximately 1/1000^{th} the size of the complete library, allowing analysis of the sample approximately 1000 times faster than would occur by using the complete carrier library to analyze the gene.

One-Step Carrier Encoding

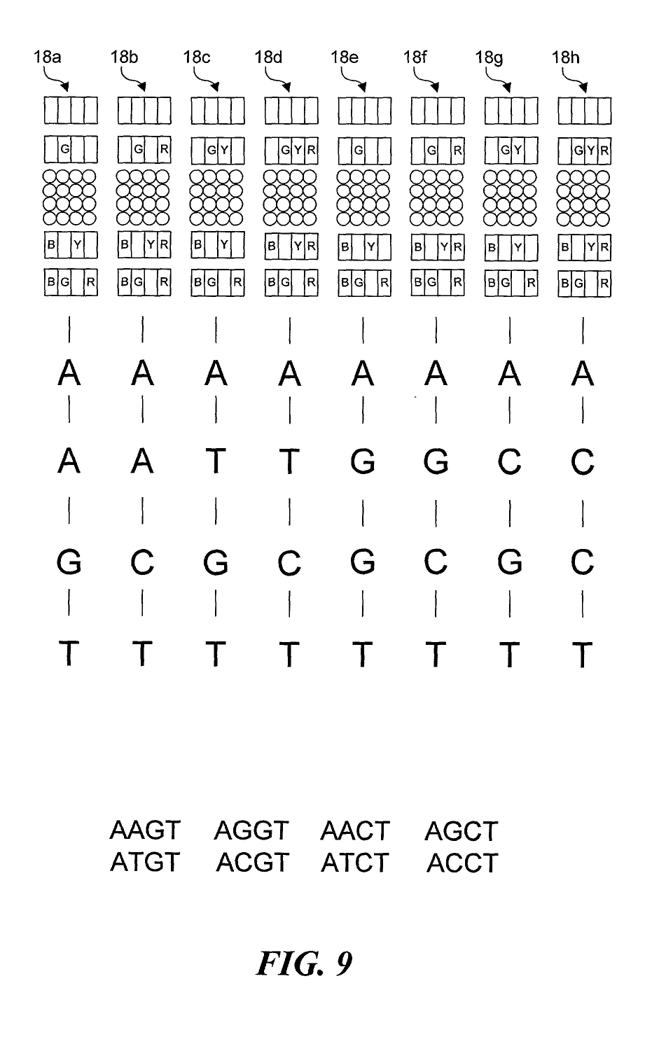

Although reporter-labeled carrier encoding can be co-executed with the synthesis of chemical compounds during the encoding process, either by SAP or directed methods, this approach can lead to interference between the encoding and synthetic processes. Accordingly, the present invention includes a method for the production of a reporter-labeled carrier library by the addition of all required reporter types to a carrier in a single step, prior to the synthesis or addition of chemical compounds to the carriers. In the present invention, instead of sequentially adding unique reporters (or several copies of the same unique reporter) to the carrier in separate steps, all reporters used to uniquely encode a carrier are added in one step. FIG. 9 illustrates this process, wherein each reaction vessel 18a-18h contains a unique combination of different reporters. Carriers are added to each reaction vessel and caused to bind to the reporters by one of a variety of different methods well known to those skilled in the art, including covalent and or non-covalent bonding using different surface functionalities on the carriers and reporters. Because each unique carrier resides in a different reaction vessel, it is possible to perform specific chemical addition or synthesis on each carrier surface after carrier encoding.

For example, FIG. 9 depicts the directed synthesis of the specific DNA library of FIG. 8 using a single-step version of the carrier encoding scheme of FIG. 6. In the present invention, the number of unique combinations that can be generated using this single-step carrier encoding approach is dictated by equation (3), as in the other examples. However, in the single step encoding process, the number of reaction vessels required is equivalent to the number of unique reporter-carrier assemblies generated. A significant advantage of the present invention is that since no chemical compounds are attached to the beads during the encoding process, a large manufacturing run of a single set of uniquely encoded beads can be used for any number of different compounds, which are subsequently synthesized on or attached to the beads. For example, a library of 10,000 unique beads can be created and then later used for SNP analysis wherein DNA oligomers are subsequently bound to the beads. The same set of beads can alternatively be used in a multiplexed drug discovery assay in which 10,000 different compounds are bound to the beads, and the set of beads is exposed to numerous drug targets. In these examples, a cross reference table or other means may be created to correlate bead signature to compound identity. During synthesis or binding of compounds to the beads, a cross reference table is created and subsequently used to determine compound identity during or after performing the assay.

Decoding Encoded Carriers

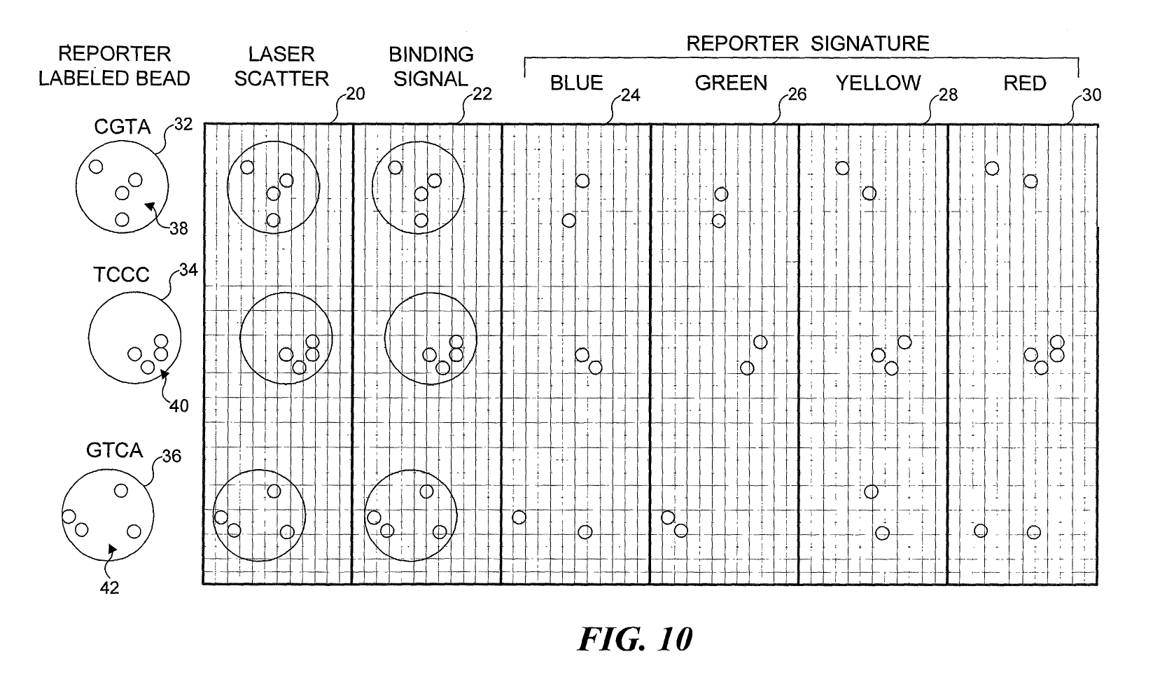

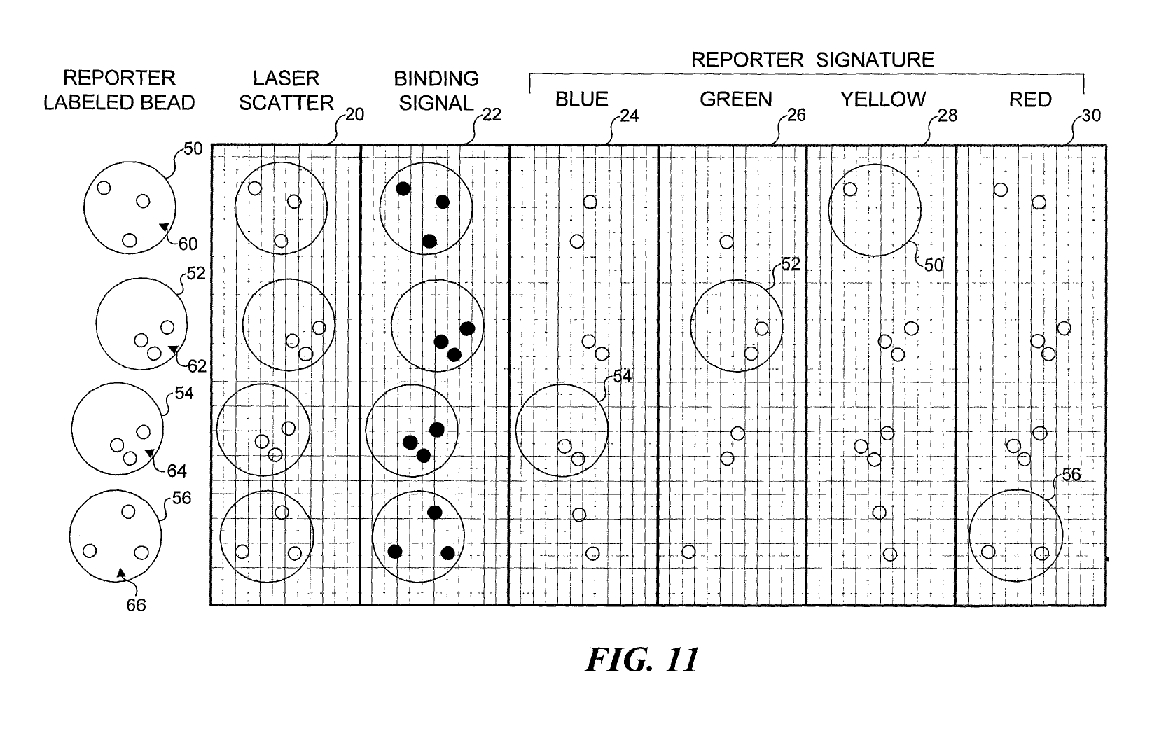

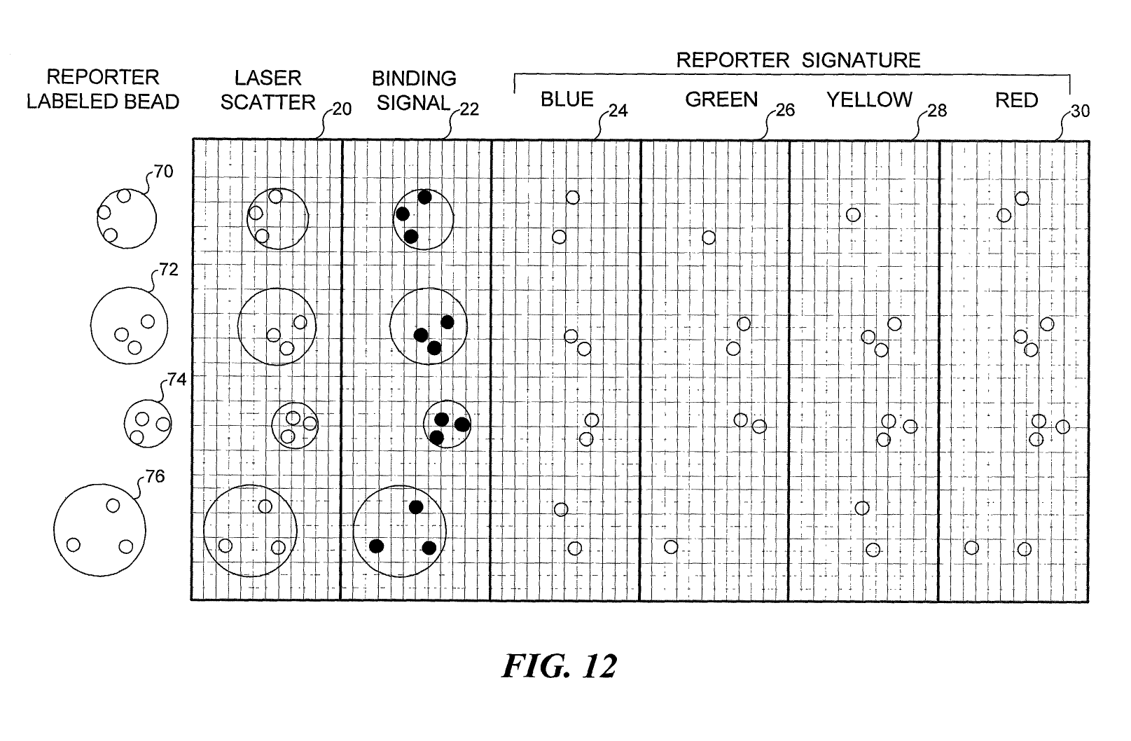

Encoded carriers can be imaged and decoded with high speed and efficiency using a flow imaging system as described in the above-referenced U.S. provisional patent application, entitled “Method And Apparatus for Synthesizing and Reading Reporter Labeled Beads.” FIG. 10 illustrates the spectral decomposition scheme by which encoded carriers are decoded when different carriers have no distinctive optical properties. Each reporter image is dispersed laterally on the detector, which is divided into a scattered laser zone 20, a binding signal zone 22, and color zones 24, 26, 28, and 30. The combination of zones that contain an image of a reporter indicate the colors with which that reporter is labeled. FIG. 10 shows three carriers 32, 34, and 36 and their associated DNA oligonucleotide sequences based on the encoding scheme illustrated in FIG. 1, as indicated by their corresponding sets of reporters 38, 40, and 42. By contrast, FIG. 11 shows the same reporter colors for each carrier, but encoded in the manner of the present invention, wherein different carriers can have optically distinguishable characteristics. In FIG. 11, the different carriers 50, 52, 54, and 56 are fluorescently labeled with different colors and respectively include reporter sets 60, 62, 64, and 66. Therefore, an image of each carrier appears in different color channels. Finally, FIG. 12 illustrates the use of carriers or substrates 70, 72, 74, and 76 that employ size as an encoding parameter in addition to the bound color coding reporters of the previous examples.

In FIG. 12, a smallest substrate 74 encodes an “A” in the first position of the oligo, a second largest substrate 70 encodes a “C” in the first position, a third largest substrate 72 encodes a “T” in the first position, and a largest substrate 76 encodes a “G” in the first position. Color reporters are also employed on the substrates. From the preceding discussion, it will be evident that any optically-detectable parameter can be used for encoding, including size, shape, intensity, polarization, etc.

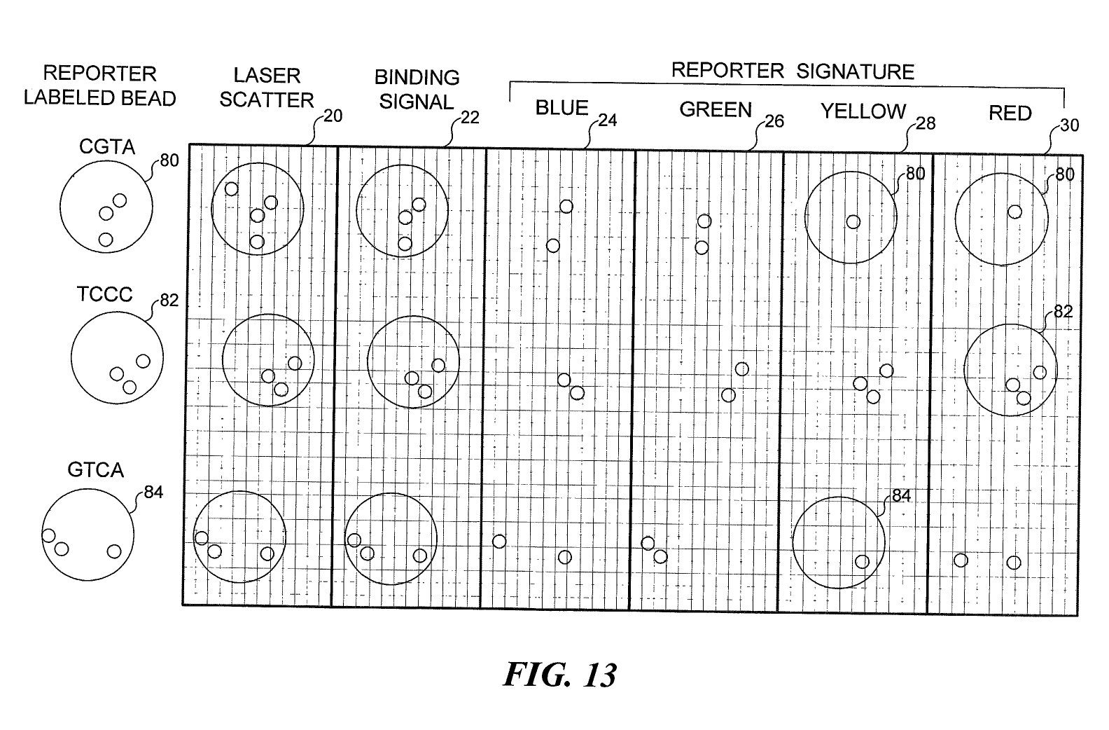

FIG. 13 illustrates images that are projected onto a detector for the spectral decomposition embodiment when three carriers are in view. In this example, a carrier 80 is distinguished by having a red and a yellow color signature, while carrier 82 has a red color signature, and carrier 84 has a yellow color signature.

Cluster Reporters

Yet another advantage of the present invention is the method of using cluster reporters. In the prior art, reporters typically take the form of small particles, each of which is labeled with one or more fluorescent compounds. However, in the present invention reporters can additionally take the form of clusters of small, singly-labeled particles. If the size of the reporter cluster is comparable to the resolution limit of the imaging system used to analyze the bead library, such as that described above and in connection with FIGS. 10-12, the cluster will be indistinguishable from a single, multiply-labeled reporters such as those described in the prior art. For example, a typical flow imaging system or fluorescence microscope has a spatial resolution limit of approximately 0.5 microns. If six singly-dyed microbeads of 0.08 micron diameter are clustered in any geometrical arrangement, through the microscope, they will appear as a single point source of up to six colors. Such singly-labeled microbeads are available commercially from a number of sources (Molecular Probes, Bangs Labs, etc.) in a wide variety of colors, materials (latex, polystyrene, silica, etc.), and with a wide variety of chemical functionality (carboxy-, amino-, avidin/biotin, etc.), typically for the convenient linkage of the microbeads to various molecules or to each other by means well known to those skilled in the art. Reporter sets can therefore be readily synthesized from commercially available microbeads or other very small particles such as quantum dots prior to their use in a combinatorially labeled bead library. One advantage of cluster reporters is that fluorescent dyes with different spectral characteristics remain isolated from each other due to their encapsulation in different singly-dyed microbeads. This isolation prevents dye quenching or resonant energy transfer due to different dye molecules residing within several nanometers of each other, which is a much smaller physical scale than the size of the microbeads themselves. Another advantage of cluster reporters is that complex optical properties, such as the presence of several colors, can be generated by assembling several microparticles, each of which has a single property.

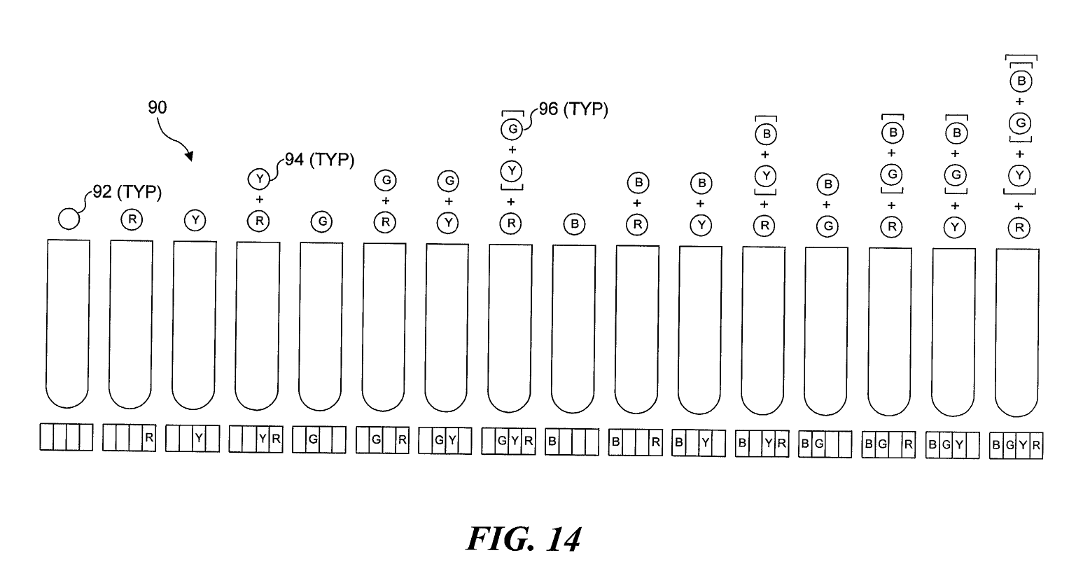

FIG. 14 illustrates one method of combining four color species (shown as B, G, Y, and R, for blue, green, yellow, and red, respectively) of singly-labeled microbeads to produce all possible binary color codes in 2⁴reaction vessels 90. Each reaction vessel is designated by the final color code of the reporter it will contain (by the colors indicated in the blocks below the reaction vessels). To each vessel is added a functionalized, singly-labeled microbead 92 (typical). If a reporter requires an additional color, the appropriately labeled microbeads 94 (typical) with complementary chemical functionality are added to the vessel for chemical binding to the first microbead species. By using complementary chemistry between microbead species (e.g., one species with carboxy-functionality and another with amine-functionality), microbeads are prevented from binding to members of their own species. The reaction can be allowed to proceed between the two microbead species until nearly all of the microbeads are reacted or the reaction can be interrupted and the unclustered microbeads filtered from the clusters. If a further reporter color is required, it is added to the reaction after the previous pairwise reaction is complete, which is illustrated in FIG. 14 by the bracketing of microbead pairs 96 (typical) above each reaction vessel. This pairwise reaction process allows the use of complementary chemistry and is much more kinetically favorable for the production of multiply-labeled reporters than reacting all the singly-labeled microbeads necessary for a reporter signature at one time in the same vessel. Alternately, microparticles of different physical properties and different optical properties can be added at different steps to facilitate the separation of unreacted microparticles between additions. For example, high density green microparticles can be added to low density red microparticles to form red-green clusters. Clusters of red and green will have intermediate density and can be separated from the remaining individual high density green and low density red microparticles by density gradient centrifugation. The subsequent addition of a high or low density blue microparticle can again be followed by separation of intermediate density red-green-blue clusters from individual blue microparticles by density gradient centrifugation.

Although the present invention has been described in connection with the preferred form of practicing it, those of ordinary skill in the art will understand that many modifications can be made thereto within the scope of the claims that follow. Accordingly, it is not intended that the scope of the invention in any way be limited by the above description, but instead be determined entirely by reference to the claims that follow.

The invention in which an exclusive right is claimed is defined by the following:

1. A method of constructing a library of optically distinct reporter labeled carriers, said method comprising the steps of:
(a) providing a plurality of carriers;
(b) providing a plurality of reaction vessels, such that at least one reaction vessel is available for each unique member of the library to be constructed;
(c) providing a plurality of optically distinct reporters;
(d) in each reaction vessel, apportioning at least one carrier and at least one reporter in a predetermined unique combination; and
(e) attaching said at least one reporter to said at least one carrier in each reaction vessel, by at least one of a physical attachment and a chemical attachment.

2. The method of claim 1, wherein at least one reaction vessel contains a carrier that is optically distinct from others of said plurality of carriers in other reactions vessels, and wherein no reaction vessel contains a mixture of optically distinct carriers.

3. A method of constructing a library of reporter labeled carriers, said method comprising the steps of:
(a) providing a plurality of singly labeled micro-particles, each singly labeled micro-particle comprising a uniquely identifiable characteristic;
(b) determining a number of unique reporters required to completely encode a desired bead library, based on the uniquely identifiable characteristics of said plurality of singly labeled micro-particles;
(c) providing a plurality of separate reaction vessels, including one reaction vessel for each unique reporter signature required;
(d) apportioning said singly labeled micro-particles among the plurality of reaction vessels, such that each reaction vessel contains at least one singly labeled micro-particle required to generate a unique reporter signature associated with that reaction vessel;
(e) for each reaction vessel requiring additional singly labeled micro-particles to generate a unique reporter signature associated with that reaction vessel, adding appropriate singly labeled micro-particles having a complementary chemistry until substantially all singly labeled micro-particles in that reaction vessel have combined; and
(f) repeating step (e) in a stepwise fashion until each reaction vessel contains either a singly labeled micro-particle having a unique reporter signature associated with that reaction vessel, or a combination of singly labeled micro-particles having a unique reporter signature associated therewith.

4. The method of claim 3, wherein said micro-particle comprises one of a quantum dot and a micro-bead.

5. The method of claim 3, wherein the uniquely identifiable characteristic comprises color.

6. The method of claim 3, further comprising the step of using a contents of each reaction vessel to combinatorially generate said desired labeled bead library.

7. The method of claim 3, further comprising the step of selecting the micro-particles so as to ensure that a size of a combination of singly labeled micro-particles required to generate a unique reporter signature associated with a specific reaction vessel is no larger than a resolution limit of an imaging system selected to read said desired bead library.
